(19) United States
(12) Patent Application Publication
(10) Pub. No.: US 2004/0229355 A1
(43) Pub. Date: Nov. 18, 2004

(54) Culture medium for long-term culture of hepatocytes

(75) Inventors: Ming Chen, Allen, TX (US); Iwona Stroynowski, Dallas, TX (US)

Correspondence Address: T. Ling Chwang, Suite 600, 2435 N. Central Expressway, Richardson, TX, 75080, US

(73) Assignee: Board of Regents University of Texas System, Austin, TX

(21) Appl. No.: 10/437,627

(22) Filed: May 14, 2003

(51) Int. Cl. C12N 5/08; C12N 5/02

(52) U.S. Cl. 435/370; 435/371; 435/404

(57) ABSTRACT

A culture medium, which is capable of sustaining long-term cultures of hepatocytes and liver cells. In this medium, mammalian primary hepatocytes retain highly replicative capacity and hepatic gene expression activity. The liver cells from genetically defined sources may be reproducibly immortalized without the delivery of foreign genes, such as viral oncogenes. The immortalized hepatocytes are non-tumorigenic, making them suitable for clinical and therapeutic purposes.

The work herein was supported in part by a grant from the United States Government under 5-P01-AI37818 awarded by the National Institute of Health. The United States Government may have certain rights in the invention.

### BACKGROUND

This invention relates to a culture medium and, in particular, to a culture medium supporting long-term culture of hepatocytes.

The liver performs many vital physiological and pathophysiological functions. In the United States, more than 0.01% of the population die each year from liver diseases (the seventh leading cause of disease-related deaths in the country), and an increasing number of patients await liver transplantation. Hepatocytes, or liver parenchymal cells, have long been used as model systems for drug screening, detoxification, hepatic gene delivery, and basic research on hepatocyte biology. The development of hepatocyte culture systems is of great importance in understanding the basic biology of hepatocytes and eventually using the cells for therapeutic applications, such as hepatocyte transplantation, development of bioartificial livers, and gene therapy.

Ideal hepatocyte cultures for use in research and therapy should (1) replicate in vitro and expand to a large number; (2) maintain hepatic functions; (3) remain non-transformed; (4) remain free of infectious pathogens; (5) be genetically identified; and (6) maintain tolerance to genetic manipulations. Unfortunately, currently available sources of hepatocytes do not meet these criteria. Hepatocytes isolated from animals have limited future applications in clinical treatments due to increasing concern about the risk of zoonoses, or diseases communicable from animals to humans. Isolating hepatocytes directly from human livers is not practical due to the shortage of available human liver tissue. Furthermore, genetic variations between human donors may significantly impact experimental results and clinical applications. Stem cells (hepatic or hemopoietic) are currently impractical for these purposes due to limited availability, difficulties in establishing proper culture conditions and ascertaining how to induce undifferentiated stem cells into becoming mature hepatocytes. So far, culturing hepatocyte cell lines has proved to be the most promising approach for generating useful hepatocyte lines.

There are three subgroups of hepatocyte cell lines being used today. The first uses malignant hepatocytes that are spontaneously transformed either in vivo or in vitro and are immortalized in vitro. The second uses normal hepatocytes which have been immortalized by the introduction of viral oncogenes, such as SV40-T antigen. Most human hepatocyte cell lines belong to these first two subgroups, but their transformed and tumorigenic qualities limit their potential use in therapeutic applications. The third subgroup uses normal hepatocytes, which have been immortalized spontaneously in vitro under special culture conditions. Currently only one human hepatocyte cell line, HHY41 (Kono Y., et al., Exp. Cell Res. vol. 221, pp. 478-85, 1995), and two murine hepatocyte cell lines, AML12/14 (Wu et al., Proc. Natl. Acad Sci U.S.A. vol. 91, pp. 674-78, 1994) and NMH (Wu et al., Cancer Res. vol. 54, pp. 5964-73, 1994), belong to the most desirable third subgroup of hepatocytes. However, the HHY41 line at passage 11 already showed anchorage-independent growth, a phenotype associated with cell transformation.

The AML12 line is derived from an outbred CD1 mouse strain which was made transgenic for human transforming growth factor a (“TGF-α”). Although AML12 was not transformed after culturing in vitro, the TGF-α transgenic CD1 mice show a significantly higher frequency of liver tumors, in about 75 to 85% of male mice at about 12 to 15 months of age. The cell line NMH is also derived from the outbred, non-transgenic CD1 mice. Both AML12 and NMH have been maintained in vitro for extended periods of time (more than 50 passages over a period of 15 months), and are non-tumorigenic. The AML12 line gradually loses the expression of albumin after prolonged time in tissue culture (Wu et al., Proc. Natl. Acad Sci U.S.A. vol. 91, pp. 674-78, 1994), and the albumin mRNA becomes barely detectable by reverse transcription polymerase chain reaction (“RT-PCR”) in AML12 cells maintained in laboratory conditions. NMH maintains better albumin productivity after 5 months in a serum-free medium, but this cell line requires epidermal growth factor (“EGF”) to promote cell proliferation during the first 10 months, or about 33 passages. The mechanism underlying the immortalization is not known, nor is it known whether this process is reproducible. Furthermore, the genetic background of the CD1 strain is not characterized, thus limiting its application for studies for which such information is critical, for example, the study of transplantation into inbred laboratory mice or immune response generated in CD1 mice to transplanted AML12 or NMH lines.

While the third subgroup of the normal human or mouse hepatocyte cell lines is most desirable for therapeutic applications, it has proven difficult to maintain in long-term cultures. Even under the most optimal conditions, human primary hepatocytes undergo in vitro less than 10 population doublings, corresponding to a viable culture period of only a few days to a few weeks, and differentiated cell functions are lost over a period of days.

Wu et al. (Cancer Res. vol. 54, pp. 5964-73, 1994) developed a serum-free medium for culturing CD1 mouse hepatocytes of the NMH line described above. This medium, consisting of Dulbecco's modified Eagle's medium, DMEM/Ham's F12 supplemented with insulin, transferrin, dexamethasone, nicotinamide and selenium, required the addition of EGF in order to promote cell growth and replication. The necessary proportions of each component of the medium are not clearly defined.

In addition, another growth medium has been developed for the primary culture of human hepatocytes (Roberts et al., Hepatology vol. 19, pp. 1390-99, 1994; Kono et al., supra). This medium utilizes α-MEM as a basal medium with supplements including serum, linoleic acid, bovine serum albumin (“BSA”), glucagon, thyrotropin-releasing factor, proline, selenite, nicotinamide, hydrocortisone, insulin and EGF. The long-term cultures were established using this growth medium in combination with, on one occasion, co-culture with rat liver epithelial cells and supernatants from HepG2 or HH09 hepatocyte lines (Roberts et al.), and, on another occasion, a collagen gel sandwich culture system (Kono et al.). The former system requires a co-culture of rat epithelial cells and conditioned media, and the latter system emphasizes the importance of the collagen sandwich system during the primary culture. Block et al. (U.S. Pat. No. 6,043,092; J. Cell. Biol. vol. 132, pp. 1133-49, 1996) have reported a chemically defined medium for hepatocyte population expansion and clonal growth. This medium includes a basal medium complemented with several growth-promoting components, such as nicotinamide, transferrin, insulin, dexamethasone, amino acids, trace metals, and simple carbohydrates. The populations of hepatocytes cultured in this medium quickly lose expression of hepatocyte-specific genes such as albumin and cytochrome P450IIB1 in one to two weeks and acquire expression of markers expressed by bile duct epithelium, such as cytokeratin 19. Furthermore, it is not clear how long the cells can be passaged in this medium.

An extended period of culture has also been achieved with a hepatocyte growth medium (“HCGM”) (Tateno, et al., Am. J. Pathol. vol. 149, pp. 1593-605, 1996), in which rat hepatocytes divided more than 10 times over a period of 105 days. The replicative cells were from small bipotent hepatocytes whose origin has not been clarified. The cell growth was dependent on the co-culture of contaminating stellate cells.

Recently, Runge et al. (Biochem. Biophys. Res. Commun. vol. 269, pp. 46-53, 2000) developed a human hepatocyte maintenance medium (“HHMM”), consisting of DMEM and MEM supplemented with albumin, galactose, proline, selenite, transferrin, trace metals, insulin, dexamethasone, hepatocyte growth factor (“HGF”), and EGF. This medium supported the primary culture of hepatocytes in a differentiated status for up to 48 days. This study did not address the potential for continuing replication under these conditions.

Thus, there is a need to have a culture medium reproducibly capable of long-term culture of hepatocytes that remain not tumorigenic, remain free of infectious pathogens, maintain tolerance to genetic manipulations, and retain high replicative capacity as well as differentiated gene expression activity.

### SUMMARY

One embodiment of this invention concerns a culture medium for the long-term culture of mammalian hepatocytes or other liver cells. Long-term culture refers here to the proliferation of the cultured cells in vitro for a period of at least 5 months. The hepatocytic status of the cultured cells is verified by several independent criteria. The criteria include: polygonal epithelial morphology with cananicular-like structures observed by light microscopy, expression of multiple hepatic markers detected by a combination of gene array, RT-PCR, Northern blot and protein assays (for albumin, α1-antitrypsin, transferrin and hepatocytic-specific class I MHC Q10). The medium, “CM-medium,” contains a mixture of several components. The bulk of CM-medium is made up of liquid basal media formulated for mammalian cell cultures, such as Dulbecco's Modified Eagle Medium (“DMEM”) and Ham's F12 Medium, alone or in combination. These may be purchased in either liquid or powder forms or self-prepared. An optional serum, such as fetal bovine serum (“FBS”), is added to improve colony formation. In addition, growth factors including, but not limited to, insulin or transferrin may be added to enhance proliferation. A mineral component, such as seleneous acid, is also present to inhibit apoptosis. A corticosteroid, such as dexamethasone, is added as an anti-inflammatory agent. A niacin compound, such as nicotinamide, helps to stimulate replication and improve viability and cell function. Adding a vitamin C component, such as L-ascorbic acid, aids in colony formation and collagen synthesis. Optionally, the additional growth factor EGF may be added.

To date, eight mouse-derived independent cell lines have been continuously cultured in CM-medium. The HepB6-1 line originated from a well-studied and genetically-defined inbred mouse strain, C57BL/6J. The immortalization was spontaneous and did not involve delivery of exogenously supplied oncogenes. Six additional lines were from C57BL/6 (B6) mice and B6 mutant mice, and the final one was from another commonly used strain, BALB/cJ. In CM-medium, hepatocytes display highly replicative capacity and can grow in vitro without signs of cell senescense. This culture system thus provides a reliable source of unlimited hepatocytes and abrogates the use of tumor oncogenes for immortalization of these cells. The immortalized hepatocytes are not tumorigenic, making them ideal for developing and testing hepatocyte cell lines with therapeutic potentials. The cultured hepatocytes can be genetically manipulated by introducing or inhibiting the genes of interest as they tolerate selection procedures such as drug selection and fluorescence activated cell staining and sorting.

In addition, long-term cultured cell lines can be generated by culturing the non-parenchymal cells of mouse liver with CM-medium. The cell lines show morphological features of either hepatic stellate cells (“HSC”) or hepatic epithelial cells (stem cells).

Commercially-supplied human hepatocytes (In Vitro Technologies, MD) have also been cultured in CM-medium. By the 11^{th} day, all hepatocytes cultured in the commerically supplied Hepatocyte Incubation Medium died, while the cells in CM-medium or CM-medium lacking FBS continued to survive for up to one month. A non-hepatocytic, epithelial cell line was also generated from a primary culture of human hepatocytes with CM-medium.

### BRIEF DESCRIPTION OF FIGURES

FIG. 1 shows the quantitation of intracellular albumin protein in various cell lines by flow cytometry.

### DETAILED DESCRIPTION OF PREFERRED EMBODIMENTS

A preferred embodiment of CM-medium should contain about 60% to nearly 100% (by volume) of liquid basal medium, such as DMEM, Ham's F12 Medium, or a combination of both. The liquid basal medium is preferably present at about 80% to nearly 100% and most preferably at about 90%. In addition, a preferred embodiment contains about 0% to about 40% by volume of FBS, preferably from about 5% to about 20%, and more preferably about 10%. Also, the medium should contain about 0 μg/L to about 100 μg/L insulin, preferably about 1 μg/L to about 20 μg/L, and more preferably about 5 μg/L. Next, the medium should contain about 0 μg/L to about 100 μg/L transferrin, preferably about 1 μg/L to about 20 μg/L, and more preferably about 5 μg/L. In addition, the medium should contain seleneous acid in a concentration of up to about 1×10⁻⁶ M, preferably from about 1×10⁻⁹ M to about 1×10⁻⁷ M, and more preferably about 3.8×10⁻⁸ M. Also, the medium should contain dexamethasone in a concentration of about 1×10⁻⁸ M to about 1×10⁻⁶ M, preferably from about 3×10⁻⁸ M to about 3×10⁻⁷ M and more preferably about 1×10⁻⁷ M. Next, the medium should contain about 1×10⁻³ M to about 1×10⁻¹ M nicotinamide, preferably from about 3×10⁻³ M to about 3×10⁻² M, and more preferably about 1×10⁻² M. In addition, an ascorbic acid salt, preferably L-ascorbic acid 2-phosphate, should be present in a concentration of about 1×10⁻⁵ M to about 1×10⁻³ M, preferably from about 0.6×10⁻⁴ M to about 6×10⁻⁴ M, and more preferably about 2×10⁻⁴ M. Optionally, EGF can be added in an amount of about 500 ng/L to about 50 μg/L, preferably from about 1 μg/L to about 20 μg/L, and more preferably about 5 μg/L.

### EXAMPLE 1

### Hepatocytes Cultured in CM-Medium

Murine hepatocytes were prepared using a two-step collagenase perfusion procedure (Seglen, Methods in Toxicology vol. 1, pp. 231-242, 1993). Freshly isolated hepatocytes were plated onto collagen-coated flasks at a density of 10⁵ cells/cm² and cultured thereafter with CM-medium.

The hepatocytes began to show synchronous growth, with a population doubling time of about 30 hours, after about 3 months of in vitro culture (or four to five passages). The morphological features of the cultured cells were well-differentiated, especially when EGF was transiently removed from the CM-M. One of the established cell lines being used, HepB6-1, was continuously cultured for 13 months without any signs of cell senescence. Seven other lines, from C57BL/6J (B6), B6 mutant and BALB/cJ mice, were also generated using CM-medium. The length of the culture period (to date) of each line is illustrated in Table 1. The results demonstrated that such hepatocyte long-term cultures can be reproducibly generated from freshly isolated murine hepatocytes of genetically distinct strains.

TABLE 1
Established Long-Term Cultured Hepatocyte Cell Lines

| Cell Line | Culture Period | | Passages |
|---|---|---|---|
| HepB6-1 | 12 | months | 55 |
| HepB6-2 | 9 | months | 27 |
| HepB6-3 | 5 | months | 7 |
| HepB6-4 | 8.5 | months | 23 |
| HepTAP^{-/-} | 5.5 | months | 13 |
| HepTPN^{-/-} | 5.5 | months | 12 |
| Hepβ₂m^{-/-} | 5.5 | months | 12 |
| HepBALB/c | 5 | months | 9 |

HepTAP^{-/-}, HepTPN^{-/-} and Hepβ₂m^{-/-} hepatocyte lines are derived from mice of H2^{b} background that are deficient for transporter associated with antigen processing 1 (“TAP1”), tapasin and β₂m, respectively.

### EXAMPLE 2

### Hepatic Gene Expression Maintained in Cultured Cells

Table 2 summarizes transcription of two hepatic gene markers, transferrin and α-fetoprotein (AFP), in CM-medium cultured hepatic lines. The transferrin mRNA was detected by RT-PCR in HepB6-1 through passage 39 (10 months) and in three other long-term cultured hepatocyte cell lines listed in Table 1. Results of transerrin and AFP transcription in liver and freshly isolated hepatocytes from B6 mice and the established mouse hepatocyte line AML12 and hepatoma line Hepa-1c1 (Hankinson, Proc Natl Acad Sci vol. 76, pp. 373-76, 1979) are included for comparison. A melanoma cell line (B78H1) is included as a non-hepatic control. AFP is transiently induced in the long-term cultures of early passages (up to P20) of CM-medium cultured lines but not in the primary tissues (Liver and Hepatocytes) or the late passages (HepB6-1/P39). Cyclophilin is a constitutive house-keeping gene control. The house-keeping genes cyclophilin and GAPDH were transcribed at constant levels throughout the culture periods (as defined by RT-PCR, Northern blot and gene arrays, arrays data not shown).

TABLE 2
Transcriptional Activity of Transferrin Hepatic Gene in Long-Term
Cultured Hepatocytes as Quantitated by RT-PCR^{a,b}

| | Time in | | | | |
|---|---|---|---|---|---|
| | culture | Passage | | | |
| | (months) | number | Transferrin | AFP | Cyclophilin |
| Liver | 0 | 0 | +++++ | − | + |
| Hepatocytes | 0 | 0 | +++++ | − | + |
| HepB6-1 | 4 | 10 | +++++ | +++ | + |
| | 7 | 20 | +++++ | ++ | + |
| | 10 | 39 | +++++ | − | + |
| HepTAP^{-/-} | 5 | 10 | +++++ | + | + |
| HepTapasin^{-/-} | 5 | 10 | +++++ | + | + |
| Hepβ₂m^{-/-} | 5 | 9 | +++++ | + | + |
| AML12 | Un^{c} | Un | +++++ | − | + |
| Hepa-1c1 | Un | Un | ++++ | + | + |
| B78H1 | Un | Un | − | − | + |

^{a}The number of + signs correlates with intensity of the amplified transferrin cDNA bands stained with ethidium bromide: + (weak); +++++ (very strong); ++, +++, ++++ (intermediate levels).
^{b}Transferrin transcripts were detected with transferrin gene-specific primers.
^{c}Un (unknown).

The transcriptional activity of serum albumin was analyzed by Northern blot hybridization (Table 3). Ten micrograms of total RNA were resolved by agarose electrophoresis and the albumin RNA was detected with a mouse serum albumin RNA probe. The serum albumin transcripts were highly expressed in the early passages of all CM-medium maintained long-term cultures (up to P10/5 months). The albumin signal decreased thereafter, but was still visible at P60/13 months (HepB6-1) upon longer exposure of film (not shown). GAPDH transcription levels are shown as a house-keeping gene controls.

TABLE 3
Transcriptional Activity of Albumin Hepatic Gene in Long-Term
Cultured Hepatocytes as Quantitated by Northern Blot Hybridization^{a,b}

| | Time in culture | Passage | | |
|---|---|---|---|---|
| | (months) | number | Albumin | GAPDH |
| Liver | 0 | 0 | +++++ | +++ |
| Hepatocytes | 0 | 0 | +++++ | +++ |
| HepB6-1 | 4 | 10 | ++ | +++ |
| | 7 | 20 | ++ | ++++ |
| | 13 | 60 | + | ++++ |
| HepTAP^{-/-} | 5 | 10 | ++++ | +++++ |
| HepTapasin^{-/-} | 5 | 9 | +++ | +++++ |
| Hepβ₂m^{-/-} | 5 | 9 | ++++ | +++++ |
| HepBALB/c | 3 | 4 | +++++ | +++++ |

^{a}The number of + signs correlates with intensity of northern hybridization signal: + (weak); +++++ (very strong); ++, +++, ++++ (intermediate levels).
^{b}Albumin transcripts were detected with an albumin gene-specific RNA probe.

The hepatic nuclear transcription factors (“HNFs”), HNF1α, HNF1β, HNF3α, HNF3β, HNF3γ, HNF4α, and C/EBPα that are critical for hepatocyte specific gene expression were detected throughout culture periods of about six to seven months (or twenty passages) by RT-PCR. Expression of HNF1α, HNF1α, and HNF3β was enhanced compared with freshly isolated B6 hepatocytes, while expression of HNF3β, HNF3γ, HNF4α, and C/EBPα decreased over time in culture. The α1-antitrypsin, H2-Q10, C-reactive protein (“CRP”) and mannose binding protein (“MBL2”), coagulation factor II and VII were also transciently detected by gene array. Class I major histocompatibility complexes (“MHCs”), including H2-K^{b}, -D^{b}, and β₂m antigens, were similarly constitutively expressed on the hepatocyte cell surfaces throughout the culture periods. The expression patterns of class Ia and Ib MHCs, as well as antigen processing and presentation genes, were also very similar to those of primary hepatocytes.

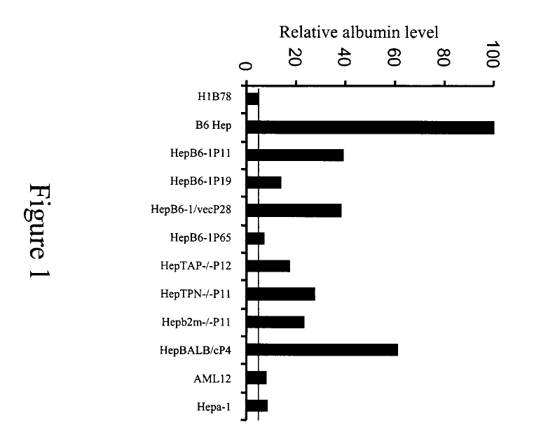

FIG. 1 shows that serum albumin, a hepatic specific protein, is produced by CM-medium-cultured hepatocytes. The intracellular albumin protein in various cell lines was quantified by flow cytometry with goat anti-mouse albumin antibodies. The freshly isolated hepatocytes (B6 Hep) are used as a reference control (100%) and the relative levels of intracellular albumin in each line are presented. HepB6-1/vecP28 is a HepB6.1 line transfected with pcDNA3.1 vector. A non-hepatic control melanoma tumor cell line (B78H1) was negative for albumin production. The horizontal line denotes the background level of staining. Hepatocytes cultured in CM-medium (HepBALB/cP4) synthesized approximately 60% of the albumin of freshly isolated hepatocytes for up to 10 weeks. The albumin levels decreased to 20 -40% of the freshly isolated hepatocytes after 4 to 7 months in culture (Passage 11 to 28, shown in HepB6-1P11, HepTPN-/-P11, Hepb2m-/-P11, HepTAP-/-P12, HepB6-1P19, and HepB6-1/vecP28). After 14 months (HepB6-1P65), the synthesis of albumin decreased further, reaching levels equivalent to that of hepatic cell lines AML12 and Hepa-1c1.

### EXAMPLE 3

### Non-Tumorigenic Phenotype of Hepatocytes Immortalized by CM-Medium

Finally, the hepatocytes cultured in the current medium did not display oncogenicity. Subcutaneous injections of 1×10⁷ HepB6-1 cells cultured in CM-medium were made in Severe Combined Immune Deficient (“SCID”) mice (B6.Cg-Foxn1^{nu}, Jackson Laboratory, ME) after 14 and 18 passages. Tumor growth did not occur. By contrast, a subcutaneous injection of 5×10⁶ cells of the known murine hepatoma Hepa-1c1 into the SCID mice did cause tumor growth by 5 weeks. In addition, all of the cells cultured in CM-medium tested negative for anchorage-independent growth in a soft agar assay at passages 18 and 58, while the control Hepa-1c1 tested positive for anchorage-independent growth under the same conditions.

### EXAMPLE 4

### Comparisons of Culture Media

Hepatocytes isolated from the BALB/cJ strain of mice were separately cultured in D/R-medium, HCGM-medium, AML12-medium, and CM-medium. The components of each culture medium are illustrated in Table 4.

TABLE 4
Comparison of Media Used for Hepatocyte Long-Term Culture

| D/R- | | | |
|---|---|---|---|
| medium | HCGM-medium | AML12-medium | CM-medium |
| DMEM & | DMEM | DMEM:Ham's | DMEM:Ham's F12 |
| RPMI | 10% FBS | F12 (1:1) | (1:1) |
| 1640 (1:1) | 10 ng/mL EGF | 10% FBS | 10% FBS |
| 10% FBS | 10 nM nicotinamide | 5 mg/L insulin | 5 mg/L insulin |
| | 0.2 mM L-ascorbic | 5 mg/L transferrin | 5 mg/L transferrin |
| | acid 2-phosphate | 3.8 × 10⁻⁸ M | 3.8 × 10⁻⁷ M |
| | 1% DMSO | selenium | selenium |
| | | 1 × 10⁻⁷ M | 1 × 10⁻⁷ M |
| | | dexamethasone | dexamethasone |
| | | | 10 nM nicotinamide |
| | | | 0.2 mM L-ascorbic |
| | | | acid 2-phosphate |

After three days, the hepatocytes in each medium showed similar morphological features. By the 13^{th} day, the cells in the D/R medium had died and the culture was terminated. The HCGM culture displayed severe reductions in the extracellular matrix and the sizes of the cell colonies. The AML12 culture lost active replication. By contrast, the CM-medium maintained culture showed highly replicative capacity and confluency of dividing cells.

After 44 days of culture, the cells in the HCGM-medium had died and the culture was terminated. The AML12-medium maintained a few non-hepatocyte-like cells that were not actively dividing and the culture was terminated. The hepatocytes in CM-medium had been passed once and were continuously dividing. After 60 days of culture, the CM-medium maintained cells had been passed twice and were actively dividing.

### EXAMPLE 5

### Subtractive CM-Media

The influence on hepatocyte growth by each individual component of complete CM-M (containing EGF and 10% FBS) was tested by culturing mouse hepatocytes with both subtractive media of CM-medium and complete CM-medium. Conclusions concerning the necessity of each individual component are shown below in Table 5.

TABLE 5
Subtractive Media Used for Hepatocyte Culture

| Subtractive | Components removed from | Necessity of the |
|---|---|---|
| Media | CM-Medium | Component |
| CM-M/I.T (−) | Insulin and transferrin | Optional |
| CM-M/S (−) | Selenous acid | Optional |
| CM-M/D (−) | Dexamethasone | Absolute |
| CM-M/EGF (−) | Epidermal growth factor | Optional |
| CM-M/FBS (−) | Fetal bovine serum | Optional |
| CM-M/N (−) | Nicotinamide | Absolute |
| CM-M/AAP (−) | L-ascorbic acid, 2-phosphate | Optional, |
| | | recommended |

By the end of the second week in culture, hepatocytes in CM-M/D (−) and CM-M/N (−) cultures were completely lost and replaced with cells of fibrous phenotype. CM-M/AAP (−) culture appeared similar to CM-M/D (−) and CM-M/N (−) but to a lesser degree. The hepatocytes in CM-M/I.T (−) and CM-M/S (−) media showed a slower growth rate compared with hepatocytes in CM-medium, while those in CM-M/EGF (−) and CM-M/FBS (−) media showed no obvious inhibition of growth. The CM-medium culture maintained highly replicative capacity. The growth patterns of the subtractive media were observed through the culture periods of 1 to 2 months. Results indicated that dexamethasone and nicotinamide were absolutely required to support the long-term growth of hepatocytes. Addition of L-ascorbic acid, 2-phosphate was optional, but recommended, to promote the hepatocyte growth. The other components were optional under each condition using subtractive CM-M media.

### REFERENCES CITED

### U.S. Patents

U.S. Pat. No. 6,043,092; Filed Mar. 18, 1996; Block.

### OTHER PUBLICATIONS

Block et al., J. Cell. Biol. vol. 132, pp. 1133-49, 1996.

Hankinson, Proc Natl Acad Sci vol. 76, pp. 373-76, 1979.

Kono et al., Exp. Cell Res. vol. 221, pp. 478-85, 1995.

Roberts et al., Hepatology vol. 19, pp. 1390-99, 1994.

Runge et al., Biochem. Biophys. Res. Commun. vol. 269, pp. 46-53, 2000.

Seglen, Methods in Toxicology vol. 1, pp. 231-242, 1993.

Tateno, et al. Am J Pathol. vol. 146, pp. 1593-1605.

Wu et al., Cancer Res. vol. 54, pp. 5964-73, 1994.

Wu et al., Proc. Natl. Acad Sci U.S.A. vol. 91, pp. 674-78, 1994.

What is claimed is:

1. A culture medium for the long-term culture of hepatocytes or liver cells, said culture medium having components comprising liquid basal medium, a growth factor, mineral, a corticosteroid, a niacin, and a vitamin C derivative.

2. The culture medium of claim 1, further comprising a serum, wherein said liquid basal medium is selected from the group consisting of DMEM, Ham's Medium, and a mixture thereof, said growth factor comprises insulin and transferrin, said mineral comprises seleneous acid, said corticosteroid comprises dexamethasone, said niacin comprises nicotinamide, said vitamin C derivative comprises L-ascorbic acid 2-phosphate, and said serum comprises FBS.

3. The culture medium of claim 2, wherein said growth factor further comprises EGF.

4. The culture medium of claim 2, wherein said components comprise the following ranges of amounts of ingredients:
(a) Liquid basal medium, in an amount from about 60% to about nearly 100% by volume;
(b) FBS, in an amount from about 0% to about 40% by volume;
(c) insulin, in an amount from about 0 μg/L to about 100 μg/L;
(d) transferrin, in an amount from about 0 μg/L to about 100 μg/L;
(e) seleneous acid, in a concentration of up to about 1×10⁻⁶ M;
(f) dexamethasone, in a concentration from about 1×10⁻⁸ M to about 1×10⁻⁶ M;
(g) nicotinamide, in a concentration from about 1×10⁻³ M to about 1×10⁻¹ M; and
(h) L-ascorbic acid 2-phosphate, in a concentration from 1×10⁻⁵ M to about 1×10⁻³ M.

5. The culture medium of claim 4, further comprising EGF in an amount from about 500 ng/L to about 50 μg/L.

6. A culture medium for the long-term culture of hepatocytes or liver cells, said culture medium having components comprising:
(a) Liquid basal medium, wherein said liquid basal medium is a mixture of DMEM and Ham's F12 medium in equal parts, in an amount from about 80% to about nearly 100% by volume;
(b) FBS, in an amount from about 5% to about 20% by volume;
(c) insulin, in an amount from about 1 μg/L to about 20 μg/L;
(d) transferrin, in an amount from about 1 μg/L to about 20 μg/L;
(e) seleneous acid, in a concentration from about 1×10⁻⁹ M to about 1×10⁻⁷ M;
(f) dexamethasone, in a concentration from about 3×10⁻⁸ M to about 3×10⁻⁷ M;
(g) nicotinamide, in a concentration from about 3×10⁻³ M to about 3×10⁻² M; and
(h) L-ascorbic acid 2-phosphate, in a concentration from 0.6×10⁻⁴ M to about 6×10⁻⁴ M.

7. The culture medium of claim 6, further comprising EGF in an amount from about 1 μg/L to about 20 μg/L.

8. The culture medium of claim 6, wherein said components comprise the following amounts:
(a) DMEM and Ham's F12 medium in equal parts, in an amount of about 90%;
(b) FBS, in an amount of about 10% by volume;
(c) insulin, in an amount of about 5 μg/mL;
(d) transferrin, in an amount of about 5 μg/mL;
(e) seleneous acid, at a concentration of about 3.8×10⁻⁸ M;
(f) dexamethasone, at a concentration of about 1×10⁻⁷ M;
(g) nicotinamide, at a concentration of about 1×10⁻² M; and
(h) L-ascorbic acid 2-phosphate, at a concentration of about 2×10⁻⁴ M.

9. The culture medium of claim 7, further comprising EGF in the amount of about 5 μg/L.

10. A method for the long-term culture of hepatocytes or liver cells, comprising the steps of:
(a) isolating hepatocytes; and
(b) plating hepatocytes in collagen-coated vessels in the culture medium of claim 1.
